(19) United States
(12) Patent Application Publication
(10) Pub. No.: US 2007/0239242 A1
(43) Pub. Date: Oct. 11, 2007

(54) APPARATUS AND METHOD FOR ANCHORING AN IMPLANTED LEAD

(76) Inventor: Daniel A. GRAUBERT, Etna, NH (US)

Correspondence Address: GARDNER GROFF GREENWALD & VILLANUEVA. PC, 2018 POWERS FERRY ROAD, SUITE 800, ATLANTA, GA, 30339, US

(21) Appl. No.: 11/697,176

(22) Filed: Apr. 5, 2007

Related U.S. Application Data
(60) Provisional application No. 60/789,970, filed on Apr. 6, 2006.

(51) Int. Cl. A61N 1/04

(52) U.S. Cl. 607/116; 607/117

(57) ABSTRACT

An anchor for a spinal cord stimulator lead or an intrathecal catheter lead includes a gripper for securely gripping the lead and a suture ring for suturing the anchor to the adjacent tissue.

### CROSS-REFERENCE TO RELATED APPLICATION

This application claims priority to U.S. Provisional Patent Application Ser. No. 60/789,970, filed Apr. 6, 2006, which is hereby incorporated herein by reference in its entirety for all purposes.

### TECHNICAL FIELD

The present invention relates generally to medical devices and related methods of use and implantation, and particularly to an apparatus and method for anchoring an implanted lead in a human or animal subject.

### BACKGROUND OF THE INVENTION

Electrical stimulation systems, including spinal cord stimulators, peripheral nerve stimulators, and bladder stimulators, typically require anchoring of a stimulator lead to the adjacent fascial connective tissues to prevent movement of the lead from the precise location that provides the best therapeutic effect. Such conventional anchoring of stimulator leads typically involves threading a flexible tube over the lead and tying sutures around the tube to cinch it down on the lead and to prevent the lead from sliding within the tube. The tube is then sutured to a secure tissue in the patient to keep it in place. However, sutures that are too tight can damage the lead, while sutures that are too loose can allow the lead to slip and migrate. Additionally, knots can come undone and allow for lead movement. Moreover, sutures, including “permanent” sutures such as silk, are subject to breakdown within the human body and may loosen over time.

Another technique involves a “snap lock” which is threaded onto the lead and snapped into a locked position, which secures the lead to the snap lock device. The snap lock is then sutured to secure tissues on the patient to keep it in place. The snap lock device is bulky and can be uncomfortable, especially on thin patients.

Thus it can be seen that needs exist for improvements to anchoring apparatuses and methods of anchoring implanted leads to provide a more secure anchor for the implanted leads within the body. It is to such provision that the present invention is primarily directed, which solves the above-described shortcomings and other related and unrelated shortcomings.

### SUMMARY OF THE INVENTION

In example forms, the system and method of the present invention provide more secure anchoring of implanted leads, more reproducible anchoring of implanted leads, and identification of the location of the anchor using x-ray technology (or other imaging technology). Additionally, the system and method of the present invention reduces operator variability/error and reduces the incidence of movement or “migration” of the implanted lead, which thus would reduce the incidence of re-operation to reposition a lead that has migrated.

In example forms, the present invention is an apparatus for anchoring an implanted lead. The apparatus includes an anchor for anchoring an implanted lead including a head portion having a base and a receiver; a flexible tongue having a first end extending from the base of the head portion and a free end extending therefrom, and configured for engagement within the receiver; and a compressible gripper for engagement of the implanted lead between the tongue and the head portion. Together, the receiver and tongue form a ratchet to prevent retraction of tongue from receiver.

In another aspect, the present invention is a method of anchoring an implanted lead. The method includes the steps of implanting an anchor having a gripper for gripping the implanted lead and a suture ring for securing the anchor to adjacent tissue; tightening the anchor against the implanted lead such that the gripper securely grips the lead; trimming excess tongue from the anchor; and suturing the suture ring to adjacent tissue.

In another aspect, the invention is an apparatus for implanting an anchor for an implanted lead. The apparatus includes means for tightening the anchor to a tension within a specified range and means for trimming an excess tongue/tail portion of the anchor. The apparatus can be disposable, or the apparatus can be reusable.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a front view of an anchor shown in a closed position and wrapped around a lead according to an example embodiment of the present invention.

FIG. 2 is a front view of the anchor of FIG. 1, shown in an open position.

FIG. 3 is a first side view of the anchor of FIG. 1, shown from the perspective of viewpoint B.

FIG. 4 is a second side view of the anchor of FIG. 1, shown from the perspective of viewpoint C.

FIG. 5 shows end views of the anchor of FIG. 1, from the perspective of views D and E.

FIG. 6 is a pictorial representation showing example placement of a percutaneous lead with the anchor of FIG. 1 in a patient.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

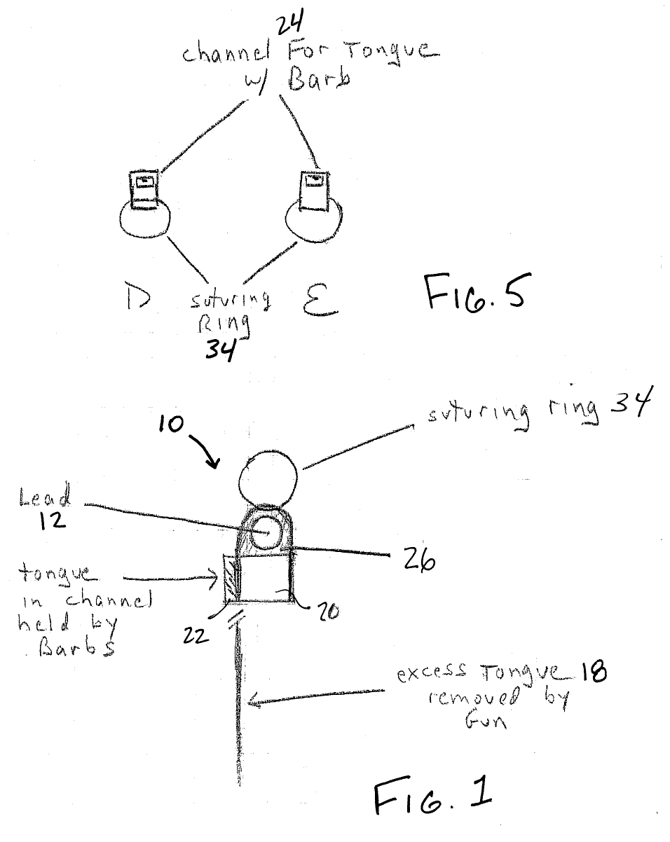

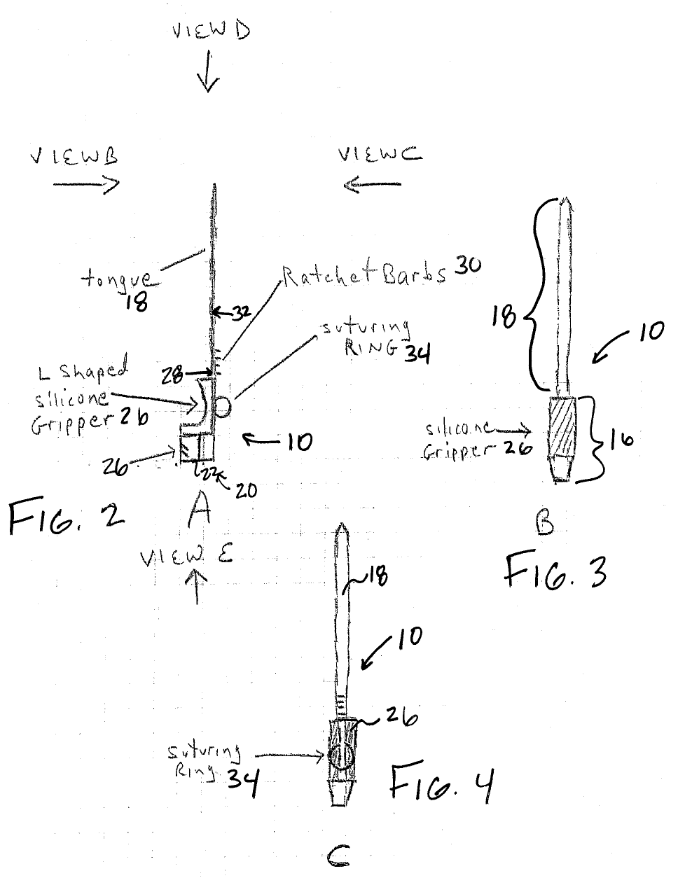

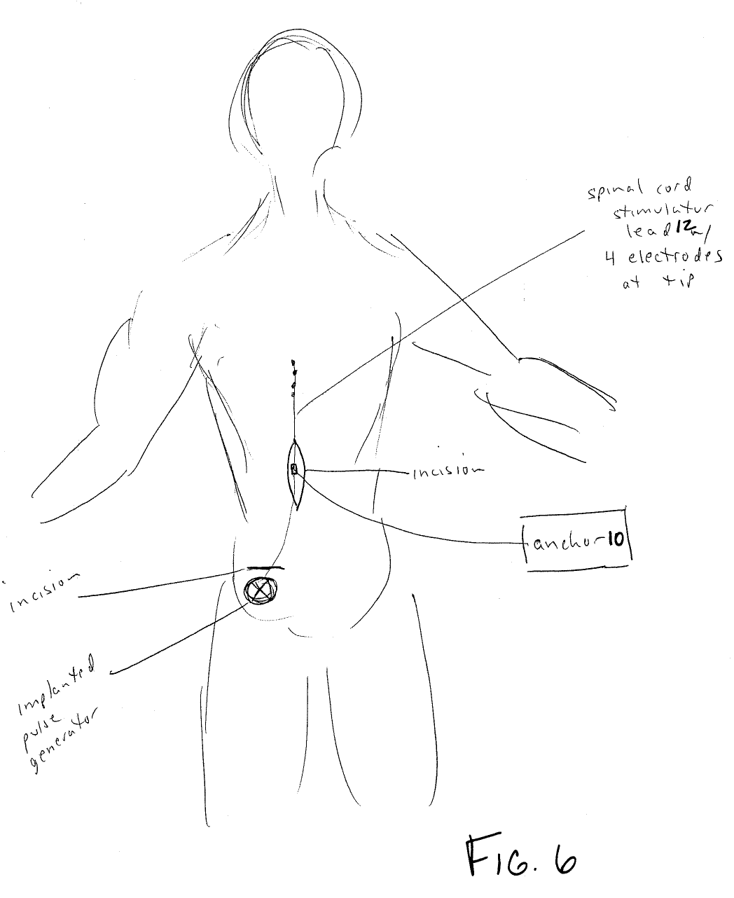

FIGS. 1-5 show an anchor 10 according to an example form of the invention for anchoring an implanted lead 12 in a human or animal subject. In example applications, the lead 12 is a percutaneous lead, a surgical, paddle-type spinal cord stimulator lead, or an intrathecal catheter lead. The anchor 10 is similar to a cable tie, which is placed over the lead 12 and tightened to a preset tightness by using a tightening device operating similar to a cable tie gun (not shown). Preferably, the anchor 10 is constructed of a flexible, biocompatible, and non-degradable material. Also preferably, the width of the anchor 10 is about a half of an inch or less, although those skilled in the art will understand that the width of the anchor can vary.

The anchor 10 further includes a head portion 16 at a proximal end thereof and a tail or tongue portion 18 extending distally from the head portion 16. The head portion 16 has a base 20 with a receiver 22 extending at a first direction. The tongue 18 of the anchor 10 extends in a second direction, perpendicular to the first direction of the receiver 22. The receiver 22 has a channel 24 for receiving the free end of the tongue 18. The receiver 22 also contains a pawl (not shown).

Preferably, the head portion 16 also includes a gripper 26 permanently affixed to an inner face 28 thereof. The gripper 26 grasps the lead 12, cushions the lead, and secures the lead in place within the anchor 10. Preferably, the gripper 26 is constructed of a biocompatible material that creates a high coefficient of friction between itself and the lead 12 when it is placed against the lead. For example, silicone can be used for the gripper 26, although those skilled in the art will understand that other biocompatible materials could also be employed. Additionally, the gripper 26 can have a generally “L-shape” when the lead 12 is in the open configuration as shown in FIG. 2 such that the gripper can bend and wrap around the lead 12 and enclose the lead within a closed loop configuration.

Preferably, the tongue portion 18 is a flexible member including a plurality of teeth or barbs 30 on an outer face 32 thereof. The tongue portion 18 is inserted through the channel 24 of the receiver 22 such that the anchor 10 forms a loop that can be tightened against the lead 12. When closed around the lead 12, the tongue 18 forms a U-shaped member with a first leg of the “U” being the part that extends from the base 20 of the head 16 and a second leg of the “U” being part of the tongue 18 inserted in the channel 24 of the receiver 22. The gripper 26 is enclosed within the U-shaped member and compressed against the lead 10 when tightened by pulling the free end of the tongue 18 through the channel 24. The pawl engages the teeth 30 of the tongue portion 18 and allows the anchor 10 to be tightened or pulled through the receiver in the forward direction, but not loosened or pulled through the receiver in the rearward direction. The teeth 30 and pawl prevent loosening after the anchor 10 is tightened to maintain compression. Thus, the receiver 22 and tongue 18 together form a ratchet to prevent retraction of tongue from receiver.

The excess tongue section 18 of the anchor 10 comes off of the head portion 16 at an offset of 90 degrees to minimize stresses on the anchor where it connects to the ratchet while making a tight circle or loop. Preferably, the excess tongue section 18 is trimmed from the anchor.

The anchor 10 preferably also includes a suture ring 34 permanently affixed to the outer surface of the head portion 16 generally behind the gripper 26. The ring 34 serves as a loop to secure the anchor 10 to the adjacent tissues using a suture. Preferably, the ring 34 is constructed of a biocompatible metal, such as stainless steel or titanium, such that the ring can be viewed with an x-ray or other imaging technology. Alternatively or additionally, the ring 34 (or another portion of the anchor 10) may include a radio-opaque marker for identifying the location of the ring, and hence the location of the lead, in the body of the patient using conventional imaging technology. Identifying the location of the ring and lead can be useful both when implanting the lead and anchor and when performing additional revision surgeries of the stimulator system.

A tool similar to a cable tie gun (not shown) can be used to tighten the anchor 10 to a preset tightness that will avoid damage to the lead 12 while still holding it securely. The tool also serves to cut off excess length of the tongue 18. Preferably, the tool is reusable, and thus is constructed of materials that are autoclavable or otherwise capable of sterilization. For example, the tool can be constructed of stainless steel. Alternatively, the tool is disposable. The tool preferably applies a consistent, user-independent desired tension within a predetermined safe and effective range. For example, the tool can employ a precisely calibrated spring that controls how much tension is applied by the gun, although those skilled in the art will understand that various other methods or mechanisms to control the amount of tension can be used as well.

In an example method of operation, a practitioner makes an incision on a patient's back and implants a lead 12, such as a percutaneous lead or a surgical lead for spinal cord stimulation or an intrathecal lead for drug delivery, through the incision according to standard medical procedures. Once implanted, the practitioner places the anchor 10 around the lead 12 such that the gripper 26 loosely grips the lead and the tongue portion 18 is inserted into the channel 24 of the receiver 22. Preferably, the anchor 10 is positioned and secured in an area located near the incision, as shown in FIG. 6. The practitioner uses the tightening tool to tighten the anchor 10 around the lead 12 and to trim the excess tongue 18 of the anchor. Alternatively, the practitioner can manually tighten the anchor 10 and trim excess tongue 18 with a blade or other cutting tool. The practitioner sutures the ring 34 of the anchor 10 to adjacent tissue. The practitioner secures the lead 12 to an implanted pulse generator or intrathecal pump, typically implanted in the patient's adipose tissue, and closes the incision.

While the invention has been described with reference to preferred and example embodiments, it will be understood by those skilled in the art that a variety of n the scope of the invention, as defined by

What is claimed is:

1. An anchor for anchoring an implanted lead, comprising:
a head portion having a base and a receiver;
a flexible tongue having a first end extending from the base of the head portion and a free end extending therefrom, and configured for engagement within the receiver; and
a compressible gripper for engagement of the implanted lead between the tongue and the head portion.

2. The anchor of claim 1, wherein the receiver and the tongue form a ratchet to prevent retraction of tongue from receiver.

3. The anchor of claim 2, wherein the ratchet comprises a pawl within the receiver and a plurality of barbs on the tongue.

4. The apparatus of claim 1, further comprising a suture ring affixed to an outer surface of the head portion proximate the gripper.

5. A method of anchoring an implanted lead, comprising:
implanting an anchor having a gripper for gripping the implanted lead and a suture ring for securing the anchor to adjacent tissue;
tightening the anchor against the implanted lead such that the gripper securely grips the lead;
trimming excess tongue of the anchor; and
suturing the suture ring to adjacent tissue.

6. An apparatus for implanting an anchor for an implanted lead, comprising:
means for tightening the anchor to a tension within a specified range; and
means for trimming an excess tongue/tail portion of the anchor.

7. The apparatus of claim 6, wherein the apparatus is disposable.

8. The apparatus of claim 7, wherein the apparatus is reusable.
